(19) United States
(12) Patent Application Publication
(10) Pub. No.: US 2021/0179591 A1
(43) Pub. Date: Jun. 17, 2021

(54) NEW COMPOUNDS FOR USE AS A THERAPEUTICALLY ACTIVE SUBSTANCE AND IN PARTICULAR FOR USE IN THE TREATMENT OF TUMORS

(71) Applicant: ETH ZURICH, Zürich (CH)

(72) Inventors: Blaise CALPE, Zürich (CH); Wilhelm KREK, Zürich (CH); Gisbert SCHNEIDER, Zürich (CH); Petra SCHNEIDER, Zürich (CH)

(21) Appl. No.: 16/769,880

(22) PCT Filed: Dec. 5, 2018
(86) PCT No.: PCT/EP2018/000544

§ 371 Date: Jun. 4, 2020

(30) Foreign Application Priority Data
Dec. 5, 2017 (EP) 17205416.5

(51) Int. Cl. C07D 403/14; C07D 401/14; C07D 405/14; C07D 403/12; A61K 45/06

(52) U.S. Cl. CPC C07D 403/14; C07D 401/14; A61K 45/06; C07D 403/12; C07D 405/14

(57) ABSTRACT

The present invention relates to a compound of formula (I) or a pharmaceutically acceptable salt thereof, wherein R₁ is selected from fluoro, methoxy and ethoxy, each R₂ is independently selected from hydrogen, fluorine and methyl, n is O, 1, 2, 3, 4 or 5, R₃ is selected from hydrogen, fluorine, amino, hydroxy, and a five or six membered substituted or unsubstituted ring system which may be aromatic or aliphatic, comprising 1 or 2 heteroatoms selected from the group consisting of nitrogen and oxygen, R4 is hydrogen or methyl, for use as a therapeutically active substance.

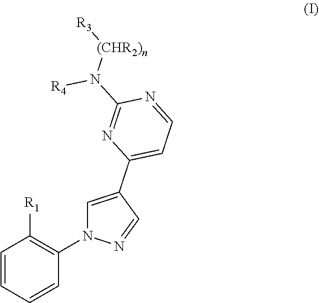

The present invention relates to compounds which act as inhibitors of dual specificity tyrosine-phosphorylation-regulated kinase 1 B (DYRK1B) (SEQ. ID. NO. 1) and are useful in the treatment of tumors. It is preferred within the present invention that the compounds also inhibit dual specificity tyrosine-phosphorylation-regulated kinase 1 A (DYRK1A) (SEQ ID NO 4).

Limited oxygen supply, called hypoxia, plays a major role in the pathobiology of tumors. This widespread phenomenon is tightly associated with tumor progression, aggressiveness and therapy resistance. Intratumoral hypoxia leads to increased activity of the hypoxia-inducible factor (HIF) family of transcription factors. HIFs regulate the expression of genes whose products contribute to angiogenesis, metabolic reprogramming, metastasis, cancer stem cell maintenance, immune evasion, and therapy resistance. Increased activity of HIFs highlights the central role of intratumoral hypoxia as a critical microenvironmental factor driving multiple key aspects of the cancer phenotype.

Moreover, hypoxic responses and consequent metabolic changes play also an important role in tumor adaption to anti-angiogenic therapy leading to the development of resistance to anti-angiogenic drugs. There are several anti-angiogenic drugs approved by the FDA which are known to invariably induce a hypoxic response that, at least in certain cases, has been shown to contribute to drug resistance and tumor relapse.

Conventional anticancer therapies target well oxygenated and proliferating cancer cells, while showing decreased efficacy against hypoxic cancer cells. Furthermore, there are no approved therapies that target hypoxic cancer cells, despite growing clinical and experimental evidence indicating that intratumoral hypoxia is a critical microenvironmental factor driving cancer progression, spread and therapy resistance.

Considerable research and clinical efforts are now directed towards identifying new targets whose inhibition would eliminate hypoxic cells and extend the benefit of anti-angiogenic therapy.

US 2014/271823 discloses that methanolic extracts of certain Carica papaya leaves have a potent inhibitory effect on HIFs.

Dual specificity tyrosine-phosphorylation-regulated kinases (DYRKs) are a subfamily of protein kinases that have dual specificity and are believed to play roles in cell proliferation and apoptosis induction. Mammalian DYRKs fall into two subgroups, class I (DYRK1A (SEQ ID NO 4) and DYRK1B (SEQ. ID. NO. 1) and class II (DYRK2, DYRK3 and DYRK4).

WO 2014/059149 discloses an inhibitor of DYRK1 activity for use in the treatment of a neoplasm in a patient.

WO 2012/098068 discloses pyrazolo[3,4-d]pyrimidines which act as inhibitors of DYRK1B (SEQ. ID. NO. 1) and/or DYRK1A (SEQ ID NO 4) and are useful in the amelioration, treatment or control of cancer, especially solid tumors, or in the amelioration, treatment or control of Down syndrome or early onset of Alzheimer's disease.

Further, Ashford et al, Biochem. J. (2014) 457, 43-56, disclose N-[2-methoxy-4-(4-methyl-1-piperazinyl) phenyl]-4-(1-methyl-1H-pyrrolo[2,3-c]pyridine-3-yl)-2-pyrimidinamine (known as AZ 191) as inhibitor of DYRK1B (SEQ. ID. NO. 1).

The problem of the present invention is to provide a compound for use as a therapeutically active substance and in particular in the treatment of tumors.

The problem is solved by the compounds according to claim 1. Further preferred embodiments are subject of dependent claims 2 to 19.

It has been found that DYRK1B (SEQ. ID. NO. 1) is essential for cancer cell survival in the context of tumor hypoxia. When functionally inhibited, DYRK1B (SEQ. ID. NO. 1) produces synthetic lethality in hypoxic cancer cells in a tumor. In particular, it has been shown that the compounds of formula (I) show a significant efficacy in the treatment of tumors. The compounds of the invention preferably also inhibit further members of the DYRK family, in particular DYRK1A (SEQ ID NO:4) including any of its isoforms.

In one embodiment, the present invention relates to compounds of formula (I)

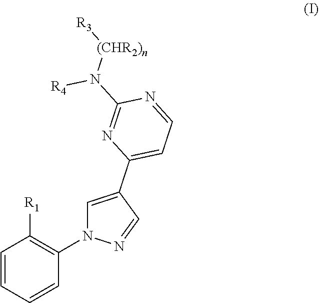

or a pharmaceutically acceptable salt thereof, wherein
R₁ is selected from fluoro, methoxy and ethoxy,
each R₂ is independently selected from hydrogen, fluorine and methyl,
n is 0, 1, 2, 3, 4 or 5,
R₃ is selected from hydrogen, fluorine, amino, hydroxy, and a five or six membered substituted or unsubstituted ring system which may be aromatic or aliphatic, comprising 1 or 2 heteroatoms selected from the group consisting of nitrogen and oxygen,
R₄ is hydrogen or methyl,
for use as a medicament, preferably for use in the treatment of tumors.

In the compounds of formula (I), R₁ is preferably fluoro or methoxy, more preferably fluoro.

R₂ is preferably hydrogen.

R₃ is preferably selected from hydrogen, fluorine, amino, hydroxy and a five or six membered substituted or unsubstituted ring system which may be aromatic or aliphatic, comprising 1 or 2 nitrogen atoms.

It is preferred that n is 0, 1 or 2 (preferably 1 or 2, more preferably 1), if R³ is the five or six membered substituted or unsubstituted ring system. Conversely, it is preferred that n is 2, 3, 4 or 5 (preferably 3, 4 or 5, more preferably 4 or 5) if R₃ is selected from hydrogen, fluorine, amino and hydroxy.

R₄ is preferably hydrogen.

In a preferred embodiment, the present invention relates to compounds of formula (Ia)

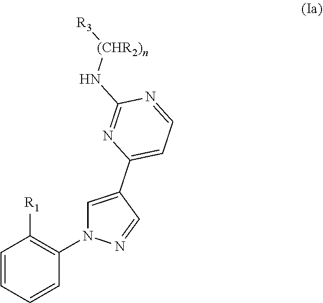

or a pharmaceutically acceptable salt thereof, wherein
R₁ is fluoro, methoxy or ethoxy,
R₂ is hydrogen or methyl,
n is 0, 1 or 2,
R₃ is hydrogen, or a five or six membered substituted or unsubstituted ring system which may be aromatic or aliphatic, comprising 1 or 2 heteroatoms selected from the group consisting of nitrogen and oxygen,
for use as a medicament, preferably for use in the treatment of tumors.

In the compound of formula (Ia), R₁ is preferably fluoro or methoxy, more preferably fluoro.

R₂ is preferably hydrogen and n is preferably 0 or 1.

R₃ is preferably a five or six membered substituted or unsubstituted ring system which may be aromatic or aliphatic, comprising 1 or 2 nitrogen atoms. If R₃ in the compound of formula (I) or (Ia) is a five or six membered ring system, it is preferably selected from the group consisting of pyridine, pyrimidine, pyrazine, pyridazine, pyrrole, furan, oxazole, isoxazole, 2H-pyran, tetrahyropyran, piperazine and pyrazole, wherein each of the listed compounds may be substituted or unsubstituted.

The term “substituted” means that the substitution can occur at one or more positions and, unless otherwise indicated, that the substituents at all substitution sites are independently selected from the specified options. The one or more substituents are preferably independently selected from halogen, such as fluorine and chlorine, methyl, ethyl, trifluoromethyl, methoxy and ethoxy. More preferably, the substituents are independently selected from the group consisting of methyl and ethyl, most preferably methyl.

Within the context of the present invention, the term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Examples for acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoroacetic acid and the like. Examples for base-addition salts include those derived from ammonium, potassium, sodium and quaternary ammonium hydroxides, such as tetramethylammonium hydroxide.

The term “treatment” of a disorder or disease as used herein (e.g., “treatment” of cancer) is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The tumor to be treated can be a solid tumor or a non-solid tumor.

Within the context of the present invention, the expression “non-solid tumors” stands for tumors that affect hematopoietic structures including components of the immune system. Examples of non-solid tumors include leukemias, multiple myelomas and lymphomas. These tumor cells generally appear in the bone marrow and peripheral circulation.

Within the context of the present invention, the expression “solid tumors” stands for primary tumors and/or metastases (wherever located) other than tumors that affect hematopoietic structures, e.g. brain and other central nervous system tumors (e.g. tumors of the meninges, brain, spinal cord, cranial nerves and other parts of the central nervous system, e.g. glioblastomas or medulla blastomas); head and/or neck cancer; breast tumors; circulatory system tumors (e.g. heart, mediastinum and pleura, and other intrathoracic organs, vascular tumors and tumor-associated vascular tissue); excretory system tumors (e.g. kidney, renal pelvis, ureter, bladder, other and unspecified urinary organs); gastrointestinal tract tumors (e.g. oesophagus, stomach, small intestine, colon, colorectal, rectosigmoid junction, rectum, anus and anal canal), tumors involving the liver and intrahepatic bile ducts, gall bladder, other and unspecified parts of biliary tract, pancreas); head and neck; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (e.g. vulva, vagina, Cervix uteri, Corpus uteri, uterus, ovary, and other sites associated with female genital organs, placenta, penis, prostate, testis, and other sites associated with male genital organs); respiratory tract tumors (e.g. nasal cavity and middle ear, accessory sinuses, larynx, trachea, bronchus and lung, e.g. small cell lung cancer or non-small cell lung cancer); skeletal system tumors (e.g. bone and articular cartilage of limbs, bone articular cartilage and other sites); skin tumors (e.g. malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

Preferably, the compound for use as a medicament, such as in the treatment of tumors, according to the present invention is selected from the group consisting of

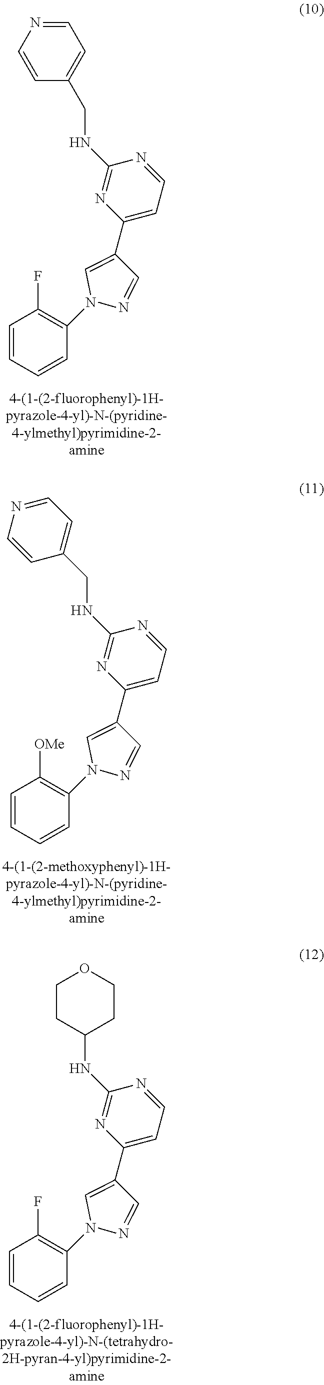 [SMILES: Fc1ccccc1-n1cc(-c2ccnc(NCc3ccncc3)n2)cn1 ; COc1ccccc1-n1cc(-c2ccnc(NCc3ccncc3)n2)cn1 ; Fc1ccccc1-n1cc(-c2ccnc(NC3CCOCC3)n2)cn1]

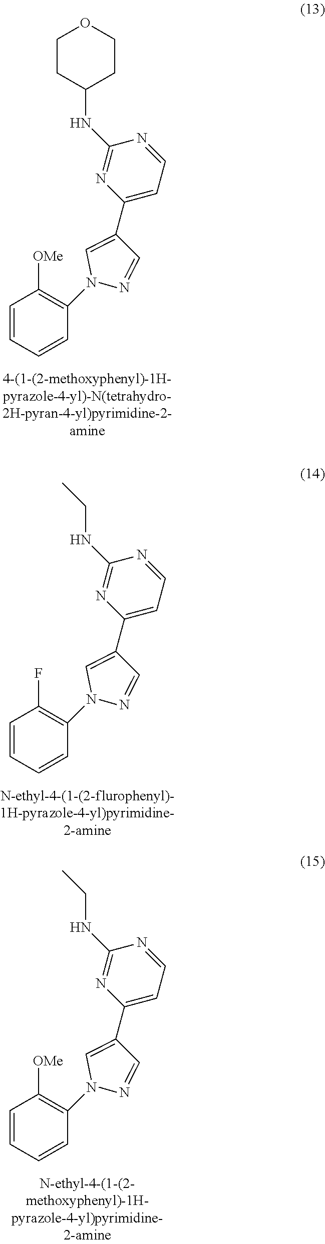 [SMILES: COc1ccccc1-n1cc(-c2ccnc(NC3CCOCC3)n2)cn1 ; CCNc1nccc(-c2cnn(-c3ccccc3F)c2)n1 ; CCNc1nccc(-c2cnn(-c3ccccc3OC)c2)n1]

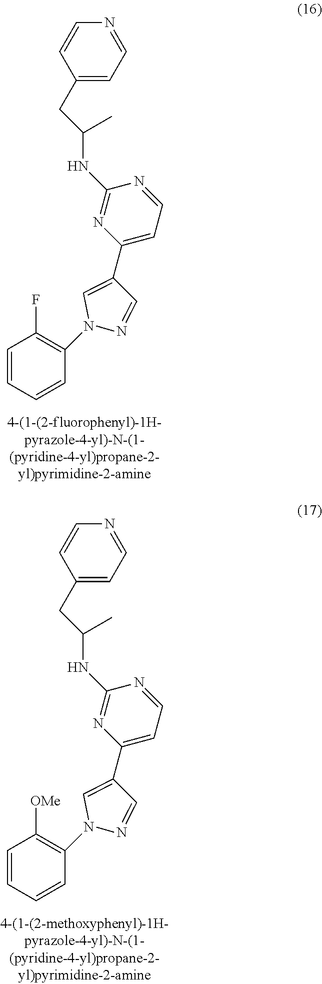 [SMILES: CC(Cc1ccncc1)Nc1nccc(-c2cnn(-c3ccccc3F)c2)n1 ; COc1ccccc1-n1cc(-c2ccnc(NC(C)Cc3ccncc3)n2)cn1]

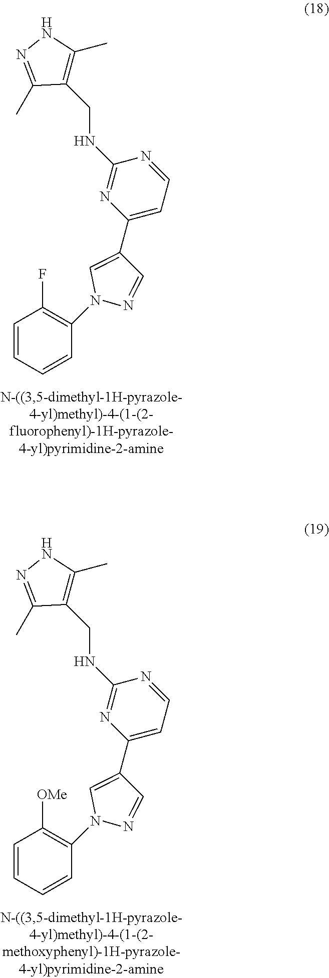 [SMILES: Cc1n[nH]c(C)c1CNc1nccc(-c2cnn(-c3ccccc3F)c2)n1 ; COc1ccccc1-n1cc(-c2ccnc(NCc3c(C)n[nH]c3C)n2)cn1]

Further preferred examples of the compound for use as a medicament, such as in the treatment of tumors, according to the present invention are selected from the group consisting of

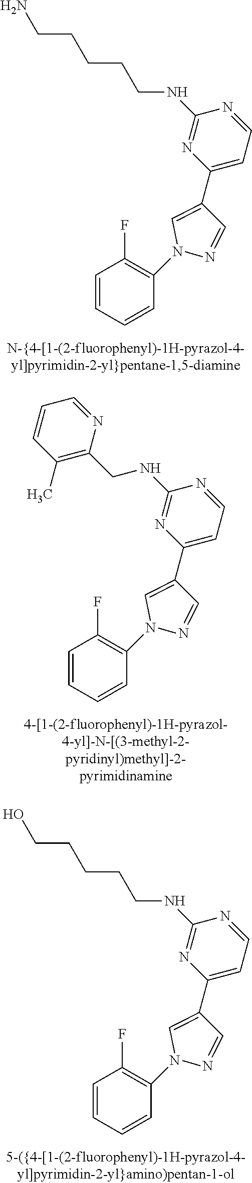 [SMILES: NCCCCCNc1nccc(-c2cnn(-c3ccccc3F)c2)n1 ; Cc1cccnc1CNc1nccc(-c2cnn(-c3ccccc3F)c2)n1 ; OCCCCCNc1nccc(-c2cnn(-c3ccccc3F)c2)n1]

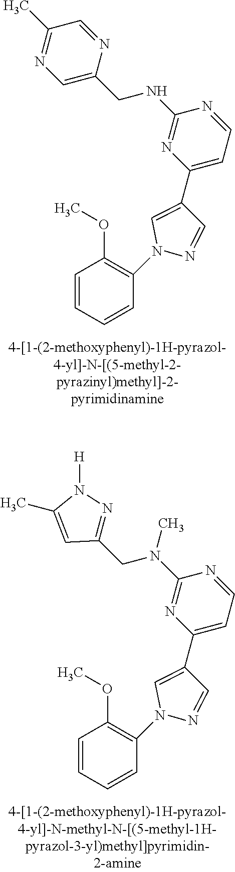 [SMILES: COc1ccccc1-n1cc(-c2ccnc(NCc3cnc(C)cn3)n2)cn1 ; COc1ccccc1-n1cc(-c2ccnc(N(C)Cc3cc(C)[nH]n3)n2)cn1]

Preferably, the compound for use in the treatment of solid or the non-solid tumors according to the present invention is selected from the group consisting of
4-(1-(2-fluorophenyl)-1H-pyrazole-4-yl)-N-(pyridine-4-ylmethyl)pyrimidine-2-amine,
4-(1-(2-fluorophenyl)-1H-pyrazole-4-yl)-N-(tetrahydro-2H-pyran-4-yl)pyrimidine-2-amine,
N-ethyl-4-(1-(2-fluorophenyl)-1H-pyrazole-4-yl)pyrimidine-2-amine,
4-(1-(2-fluorophenyl)-1H-pyrazole-4-yl)-N-(1-(pyridine-4-yl) propane-2-yl)pyrimidine-2-amine, and
N-((3,5-dimethyl-1H-pyrazole-4-yl) methyl)-4-(1-(2-fluorophenyl)-1H-pyrazole-4-yl)pyrimidine-2-amine, and most preferably 4-(1-(2-fluorophenyl)-1H-pyrazole-4-yl)-N-(pyridine-4-ylmethyl)pyrimidine-2-amine.

Other preferred compounds for use in the treatment of solid or the non-solid tumors according to the present invention is selected from the group consisting of
4-[1-(2-fluorophenyl)-1H-pyrazol-4-yl]-N-[(3-methyl-2-pyridinyl)methyl]-2-pyrimidinamine,
5-({4-[1-(2-fluorophenyl)-1H-pyrazol-4-yl]pyrimidin-2-yl}amino)pentan-1-ol,
N-(2,2-difluoroethyl)-4-[1-(2-fluorophenyl)-1H-pyrazol-4-yl]pyrimidin-2-amine, and
N-{4-[1-(2-fluorophenyl)-1H-pyrazol-4-yl]pyrimidin-2-yl}pentane-1,5-diamine, more preferably
5-({4-[1-(2-fluorophenyl)-1H-pyrazol-4-yl]pyrimidin-2-yl}amino)pentan-1-ol,
N-{4-[1-(2-fluorophenyl)-1H-pyrazol-4-yl]pyrimidin-2-yl}pentane-1,5-diamine.

Preferably, the tumor to be treated is treatment-resistant and preferably drug-resistant. Within the context of the present invention the term “treatment-resistant tumor” means the ability of tumor cells to survive and grow despite anti-cancer therapies and includes for example radiotherapy and medication intake. The term “drug-resistant tumor” means that such an anti-cancer therapy is based on medication intake. It is known that hypoxia is an environmental selection pressure that contributes to the development of therapy resistance. Thus, due to the inhibition of DYRK1B (SEQ. ID. NO. 1) by the compounds of the present invention, the micro-environment within the tumor can be significantly improved by selectively targeting hypoxic cancer cells. Consequently, the compounds according to the present invention allow overcoming therapy resistance since the remaining tumor comprises well-oxygenated and proliferating cancer cells which may be conventionally treated.

Preferably, the tumor to be treated comprises hypoxic tumor cells and/or glycolytic cancer cells. Most of the cancer cells feature increased glycolysis as a metabolic strategy. Further, also the presence of oncogenes or loss of tumor suppressor genes lead to the activation of HIF and thereby to a glycolytic phenotype. Moreover, HIF can be activated through genetic and metabolic alterations in key metabolic genes. Thus, by inhibiting DYRK1B (SEQ. ID. NO. 1) by the compounds of the present invention, tumors comprising hypoxic tumor cells and/or glycolytic cancer cells can be effectively treated. As shown in FIGS. 1a to 1e, the inhibition of the kinase DYRK1B (SEQ. ID. NO. 1) results in the cell death of the cancer cells in the hypoxic core of a multicellular tumor spheroids (MTS). In particular, the compound of formula (10) displayed a high activity against hypoxic cancer cells (FIGS. 4a to 4e) and inhibited the phosphorylation of DYRK1B substrate p27 at serine 10 in an in vitro kinase assay (FIG. 5a) and in HT29 cell cultured in hypoxia (FIG. 5b).

Preferably, the tumor comprises dormant cancer cells. Dormant or quiescent cancer cells are known to reside in hypoxic area. The above-mentioned phosphorylation of the cyclin-dependent kinase inhibitor p27 on serine 10 is linked to quiescence. In addition, dormant cancer cells are known to be often the seed of metastasis. Therefore, by inhibiting DYRK1B (SEQ. ID. NO. 1), the compounds of the present invention are particularly useful in the treatment of metastasis.

Within the present invention, “inhibiting” involves specific binding. By specific binding is meant a particular interaction between one binding partner and another binding partner, for example a compound of the present invention and a target such as DYRK1B and/or DYRK1A. Interactions between one binding partner and another binding partner may be mediated by one or more, typically more than one, non-covalent bonds. An exemplary way of characterising specific binding is by a specific binding curve. Such binding may be analysed using methods well known in the art, such as e.g. BIACORE.

In a preferred embodiment, the compounds of the present invention show inhibition of DYRK1B and/or DYRK1A to a higher extent than expected by the skilled person. The inhibition may be expressed as IC₅₀, i.e. the concentration necessary to inhibit activity to 50%. In a preferred embodiment of the present invention, the compounds have an IC₅₀ value of 12.500 nM or less, preferably 10.000 nM or less, more preferably 7.500 and even more preferably 5.000 nM or less. The IC₅₀ value may be determined using methods such as those described in the examples. As such, a suitable method comprises the steps of incubating GST-DYRK1B (e.g. at 5 nM) and DYRKtide (e.g. at 50 μM) in kinase buffer, following compound dispensing (e.g. 1 μl), e.g. 2 μl of ATP (final concentration e.g. 100 μM) and incubating, preferably at room temperature. After that, a detectable compound such as ADP-Glo™ reagent can be dispensed and incubated, preferably at room temperature. Finally, Kinase Detection Reagent can be dispensed and the solution be incubated before measuring.

Preferably, the tumor comprises tumor stem cells. Within the context of the present invention, a “tumor stem cell” means a cell which has self-replication capacity and cancer forming ability in combination and which is resistant to anti-cancer agents and/or radiation therapy and which is a causative cell of cancer recurrence. Tumor stem cells often reside in a hypoxic niche. By inhibiting DYRK1B (SEQ. ID. NO. 1) by the compounds of the present invention, the number of tumor stem cells can be significantly reduced or even the birth of tumor stem cells can be avoided. In particular leukemic cancer stem cells are very hypoxic and can be effectively treated by the compounds of the present invention.

In another embodiment of the present invention, the growth of the tumor associated with the overexpression of dual specificity tyrosine-phosphorylation-regulated kinase 1B (DYRK1B) (SEQ. ID. NO. 1) can be inhibited by the compound of the present invention. Amplification of the DYRK1B (SEQ. ID. NO. 1) occurs in many different cancers including pancreatic cancer, ovarian cancer and non-small cell lung cancer. In these cancers, DYRK1B gene acts as a potential driver oncogene, that is, as a proto-oncogene having a genetic mutation that is considered to cause a mutation specific to cancer cells and to become a main cause of cancer development. Thus, the patients can be stratified on the basis of having this amplification and then specifically be treated with the compounds of the present invention.

The present invention further relates to a pharmaceutical combination for administration in conjunction with cancer chemotherapy or radiation therapy. In particular it relates to a pharmaceutical combination comprising further an anti-angiogenic inhibitor and/or a radio- and chemotherapeutic drug, in particular when treating a tumor contains hypoxic cancer cells. In such cases, the compounds or compositions of the present invention may be administered prior to radiation therapy in order to first render cells more susceptible to such treatment. It is also envisaged to administer the compounds/compositions/combinations of the present invention when at least some of the tumor cells of a patient are in a cellcycle-arrest, e.g. due to treatment with checkpoint kinase inhibitors or Her2/EGFR inhibitors. In a further embodiment, the combination/composition comprises a compound of the invention as DYRK1B and/or DYRK1A inhibitor and a cellcycle-inhibiting compound. Such a combination/composition is preferably administered to a patient having a resistant tumor.

The term “anti-angiogenic inhibitor” stands for a substance that inhibits the growth of new blood vessels. It is known that the clinical resistance to anti-angiogenic inhibitors is quite likely due to tumor cells utilizing an alternative method for obtaining a vasculature. Further, anti-angiogenic therapy is known to induce hypoxia and thereby contribute to drug resistance. Interestingly, it could be shown that the inhibition of DYRK1B (SEQ. ID. NO. 1) inhibits the resistance to anti-angiogenic therapy. Thus, a combination medicament comprising a compound of formula (I) and an anti-angiogenic inhibitor prevents or at least diminishes the danger of a medicament resistance and allows a long-term treatment against the tumor.

Preferably, the anti-angiogenic inhibitor is selected from the group consisting of bevacizumab, erlotinib, lapatanib, sunitinib, pazopanib, imatinib, dasatanib, nilotinib, bortezomib, ibrutinib, semaxinib, vatalinib, sorafenib, leflunomide, canertinib, axitinib, nintedanib, regorafenib, pazobanib, cabozantinib, vandetanib, ziv-aflibercept, thalidomide, IMC-1C11, N-(2,3-dihydro-3,3-dimethyl-1H-indol-6-yl)-2-[(4-pyridinylmethyl)amino]-3-pyridinecarboxamide (AMG 706), 3-(4-bromo-2,6-difluoro-benzyloxy)-5-[3-(4-pyrrolidine-1-yl-butyl)-ureido]-isothiazole-4-carboxylic acid amide (CP-547,632), pazopanib (GW-786034), N-(4-(3-amino-1H-indazol-4-yl)phenyl)-N′-(2-fluoro-5-methylphenyl)urea (ABT-869), and cediranib (AXD-2171), most preferably of bevacizumab, ziv-aflibercept, sorafenib, sunitinib, axitinib, nintedanib, regorafenib, pazobanib, cabozantinib, vandetanib, and thalidomide.

The radio- and chemotherapeutic drug may be selected from the group consisting of alkylating agents, vinca alkaloids, aromatase inhibitors, selective estrogen receptor modulators, topoisomerase I inhibitors, topoisomerase II inhibitors, microtubule stabilizing and disrupting agents, tubulin binding agents, proteosome inhibitors, mTOR inhibitors and conjugated antibodies. In the pharmaceutical preparation according to the present invention, the radio- and chemotherapeutic drug is preferably selected from the group consisting of bleomycin, dactinomycin, doxorubicin, epirubicin, idarubicin, mitomycin, mitoxantron, irinotecan, topotecan, amsacrin, daunorubicin, etoposid, anagrelid, azacitidin, capecitabin, clofarabin, cytarabin, fludarabin, 5-fluorouracil, gemcitabin, mercaptopurin, nelarabin, tioguanin, cisplatin, carboplatin, oxaliplatin, bendamustin, busulfan, chlorambucil, chlormethin, cyclophosphamid, dacarbazin, ifosfamid, lomustin, melphalan, procarbazin, streptozocin, temozolomid, carmustin (-), methotrexat, pemetrexed, raltitrexed, cabazitaxel, docetaxel, paclitaxel, vinblastin, vincristin, vindesin, vinorelbin, eribulin, cladribin, podophyllotoxin, hydroxycarbamid, and ixabepilon.

Further, the compounds of the present invention are useful in increasing the sensitivity of tumor cells to radiation in radiotherapy, preferably in combination with a radiotherapeutic drug. Treatment may thus comprise treating cells with the compounds/combinations/compositions of the present invention prior to radiation therapy and/or treatment with a radiotherapeutic drug. Such treatment may be advantageous due to the higher sensitivity of cells achieved by the compounds/compositions/combinations of the present invention.

A further embodiment of the present invention is directed to the use of a compound of the present invention as inhibitor of dual specificity tyrosine-phosphorylation-regulated kinase 1B (DYRK1B) (SEQ. ID. NO. 1) and/or DYRK1A (SEQ ID NO 4), in particular since DYRK1B is a kinase which is essential for cancer cell survival in a hypoxic environment of a tumor.

It is also envisaged that the compound/combination/composition of the present invention is combined with immunotherapy. Immunotherapy may, e.g., comprise administration of an immuno-oncology agent, such as, e.g. an agent targeting CD52, PD-L1, CTLA4, CD20, or PD-1. Agents that may be used in combination with a compound/combination/composition of the present invention include, for example, alemtuzumab, atezolizumab, ipilimumab, nivolumab, ofatumumab, pembrolizumab, rituximab.

A further embodiment of the present invention is directed to an inhibitor of dual specificity tyrosine-phosphorylation-regulated kinase 1B (DYRK1B) (SEQ. ID. NO. 1) and/or DYRK1A (SEQ ID NO 4) for use in the treatment of tumors. It has been found that DYRK1B (SEQ. ID. NO. 1) is essential for cancer cell survival in the context of tumor hypoxia. When functionally inhibited, DYRK1B (SEQ. ID. NO. 1) produces synthetic lethality in hypoxic cancer cells in a tumor. Thus, by successfully inhibiting DYRK1B (SEQ. ID. NO. 1), a hypoxic environment can be reduced or avoided which allows a successful conventional treatment of a tumor both subsequently or in parallel.

A further embodiment of the present invention relates to the use of the compounds of formula I as a therapeutically active substance, and in particular for use in the treatment of disorders wherein hypoxic cells are involved. It has been surprisingly found by the present inventors that by inhibiting DYRK1A and/or DYRK1B, the compounds of the present invention are useful in the treatment of neurodegenerative diseases. Said compounds are particularly useful in the amelioration, treatment or control of Down's syndrome, Autism spectrum disorder and/or early Alzheimer's disease.

As detailed above, the compounds provided herein may be administered as compounds per se or may be formulated as medicaments, e.g. as pharmaceutical composition or combination. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers, or any combination thereof.

In particular, the pharmaceutical compositions may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-γ-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration cart be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, and vaginal.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

Alternatively, said compounds or pharmaceutical compositions can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides (see, e.g., U.S. Pat. No. 3,773,919), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(-)-3-hydroxybutyric acid (EP133988). Sustained-release pharmaceutical compositions also include liposomally entrapped compounds. Liposomes containing a compound of the present invention can be prepared by methods known in the art, such as, e.g., the methods described in any one of: DE3218121; Epstein et al., Proc. Natl. Acad. Sci. (USA) 82:3688-3692 (1985); Hwang et al., Proc. Natl. Acad. Sci. (USA) 77:4030-4034 (1980); EP0052322; EP0036676; EP088046; EP0143949; EP0142641; JP 83-118008; U.S. Pat. Nos. 4,485,045; 4,544,545; and EP0102324.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to the emulsification/spray drying process disclosed in WO 99/16419 or WO 01/85136. Spray drying of solution formulations of the compounds of the present invention can be carried out, e.g., as described generally in the “Spray Drying Handbook”, 5th ed., K. Masters, John Wiley & Sons, Inc., NY (1991), and in WO 97/41833 or WO 03/053411.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration of the compounds or pharmaceutical compositions of the present invention are oral forms of administration.

Typically, a physician will determine the dosage which will be most suitable for an individual subject. The specific dose level and frequency of dosage for any particular individual subject may be varied and will depend upon a variety of factors including the age, body weight, general health, sex, diet, mode and time of administration, rate of excretion, drug combination, the severity of the particular condition, and the individual subject undergoing therapy.

A proposed, yet non-limiting dose of the compounds according to the invention for administration to a human (of approximately 70 kg body weight) may be 0.05 to 2000 mg, preferably 0.1 mg to 1000 mg, of the active ingredient per unit dose. The unit dose may be administered, e.g., 1, 2, 3 or more times per day. The unit dose may also be administered 1 to 7 times per week, e.g., with one, two or more administration(s) per day. It will be appreciated that it may be necessary to make routine variations to the dosage depending on the age and weight of the patient/subject as well as the severity of the condition to be treated. The precise dose and also the route of administration will ultimately be at the discretion of the attendant physician.

The compounds of formula (I) can be used in combination with other therapeutic agents, including in particular other anticancer agents. When a compound of the invention is used in combination with a second therapeutic agent active against the same disease, the dose of each compound may differ from that when the compound is used alone. The combination of a compound of the present invention with a second therapeutic agent may comprise the administration of the second therapeutic agent simultaneously/concomitantly or sequentially/separately with the compound of the invention.

### BRIEF DESCRIPTION OF THE DRAWINGS

FIG. 1a shows representative fluorescence images of MTS formed with WM266-4, DLD-1 or HT29 stably transduced with shCTRL, shDYRK1B (SEQ. ID. NO. 1)#1 or shDYRK1B (SEQ. ID. NO. 1)#2. Scale bar 200 μm.

FIG. 1b shows corresponding quantification of SYTOX pixel intensity in the core of shCTRL, shDYRK1B (SEQ. ID. NO. 1)#1 or shDYRK1B (SEQ. ID. NO. 1)#2 HT29 MTS, n>30 MTS from 3 biological replicates.

FIG. 1c shows immunoblot detection of DYRK1B (SEQ. ID. NO. 1) in shCTRL, shDYRK1B (SEQ. ID. NO. 1)#1 or shDYRK1B (SEQ. ID. NO. 1)#2 HT29 MTS. γ-TUBULIN was used as loading control.

FIG. 1d shows representative fluorescence images of MTS stably transduced with shCTRL, shDYRK1B#2 or shDYRK1B #2 with DYRK1B rescue construct.

FIG. 1e shows corresponding quantification of SYTOX pixel intensity in the core of MTS, n>15 MTS from 2 biological replicates.

FIG. 1f shows an immunoblot detection of DYRK1B (SEQ. ID. NO. 1) in shCTRL, shDYRK1B #2 or shDYRK1B #2 with DYRK1B rescue construct (***p<0.001).

FIG. 2a, shows representative fluorescence images of large and small MTS HT29-HRE stably transduced with shCTRL or shDYRK1B #2. Large MTS were cultured in normoxia or incubated 18h in 1% O₂, Scale bar 200 μm.

FIG. 2b shows corresponding quantification of SYTOX intensity in the inner core of MTS, n>30 MTS from 3 biological replicates.

FIG. 2c shows tumor growth of HT29 stably transduced with shCTRL or shDYRK1B)#2 (5-7 mice/group). Nintedanib treatment started when tumor reached ˜50 mm³ (arrow). Tumor growth curves are presented as mean±SEM.

FIG. 2d shows representative tumor images and tumor weight at the end of the experiment.

FIG. 2e shows gene expression of canonical HIF target genes analyzed by qRT-PCR in nintedanib treated tumor normalized to vehicle treated tumor. (n=3 mice/group). (ns: p not significant, **p<0.01, ***p<0.001).

FIG. 3 shows compound of formula 10, 12, 14, 16 and 19 titration curve in a DYRK1B (SEQ. ID. NO. 1) in vitro kinase assay (ADP-Glo). Compounds of formula 16, 12, 14, 10 and 19 inhibited DYRK1B (SEQ. ID. NO. 1) kinase activity with an IC50 of 2 μM, 2 μM, 3 μM, 4 μM and 3 μM respectively. Titration curves show average of n=3 independent experiments, average±SD.

FIG. 4a shows representative images of MTS treated for 48 h with 5 μM of compound of formula 10 or controls. DMSO was used as negative control, AZ191 as positive control and staurosporine (STS) as a general cytotoxic control. Whole MTS was visualized by Hoechst staining and cell death by SYTOX staining. Scale bar 200 μm.

FIG. 4b shows representative images of compound of formula 10 titration in MTS.

FIG. 4c shows EC50 curve of compound of formula 10 or AZ191 activity in the core of MTS, normalized to DMSO and AZ191. Average±SD of n>10 MTS from 3 biological replicates are shown.

FIG. 4d shows representative images of small (non-hypoxic) and large (hypoxic) MTS treated with 5 μM of compound of formula 10, AZ191 or controls.

FIG. 4e shows quantification of SYTOX intensity in the core, n>30 MTS from 3 biological replicates.

FIG. 5a shows the immunoblot of an in vitro kinase assay performed with recombinant DYRK1B and p27 incubated with 100 μM ATP and increasing concentration of AZ191 or compound of formula 10.

FIG. 5b shows the immunoblot of HT29 cells cultured overnight in normoxia or hypoxia with increasing concentration of AZ191 or compound of formula 10.

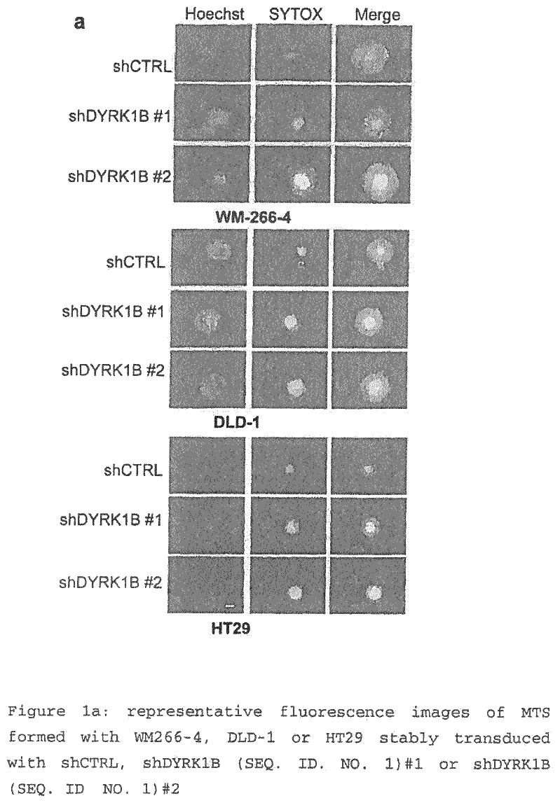

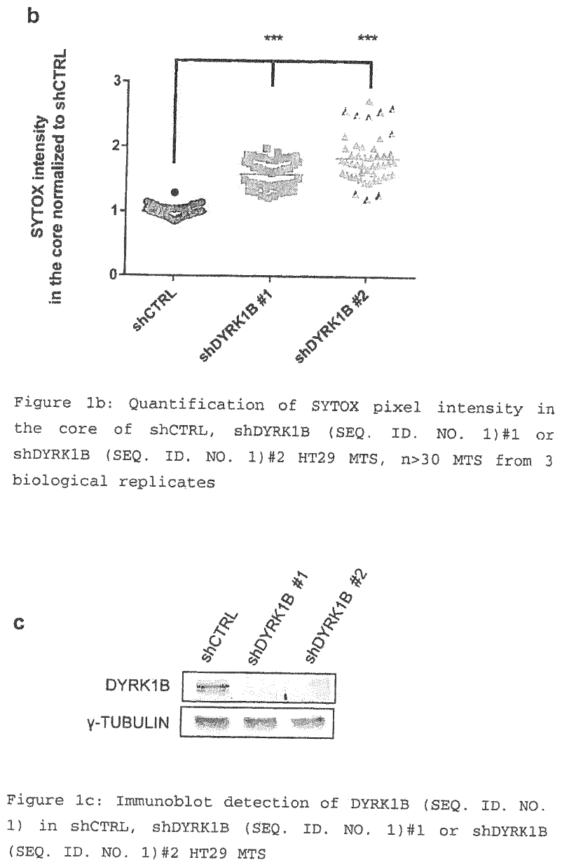

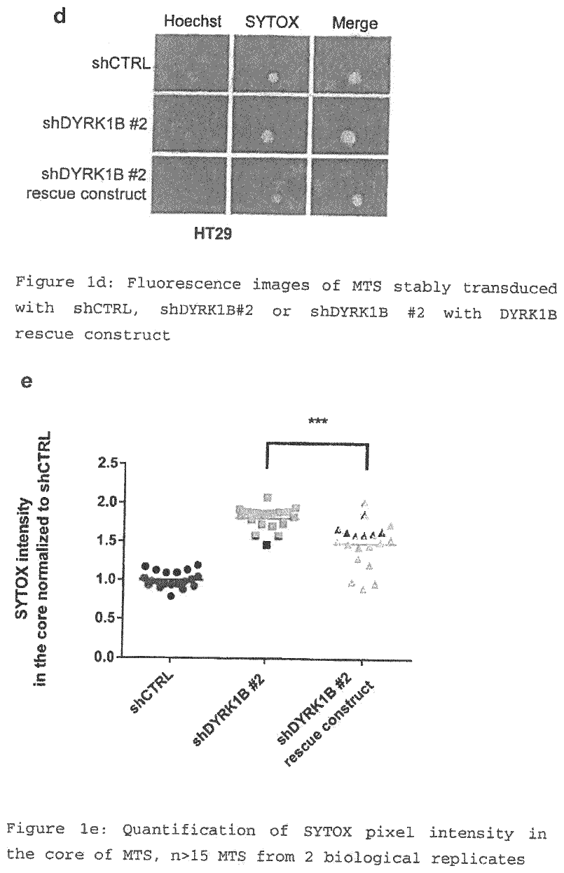

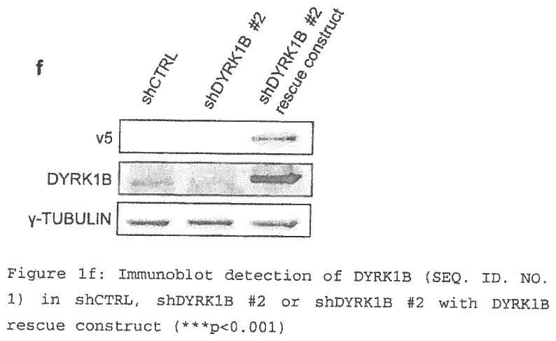

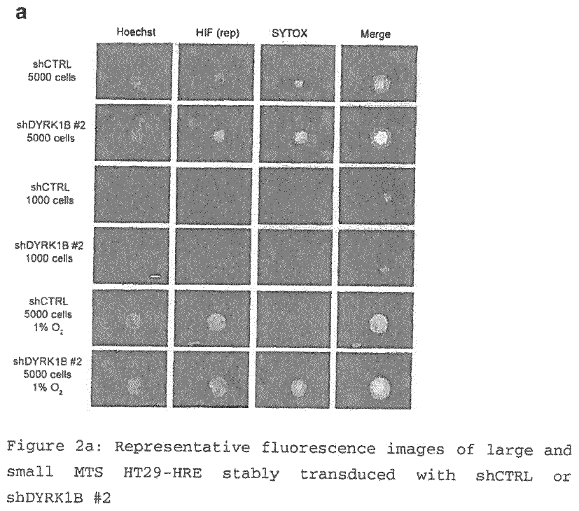

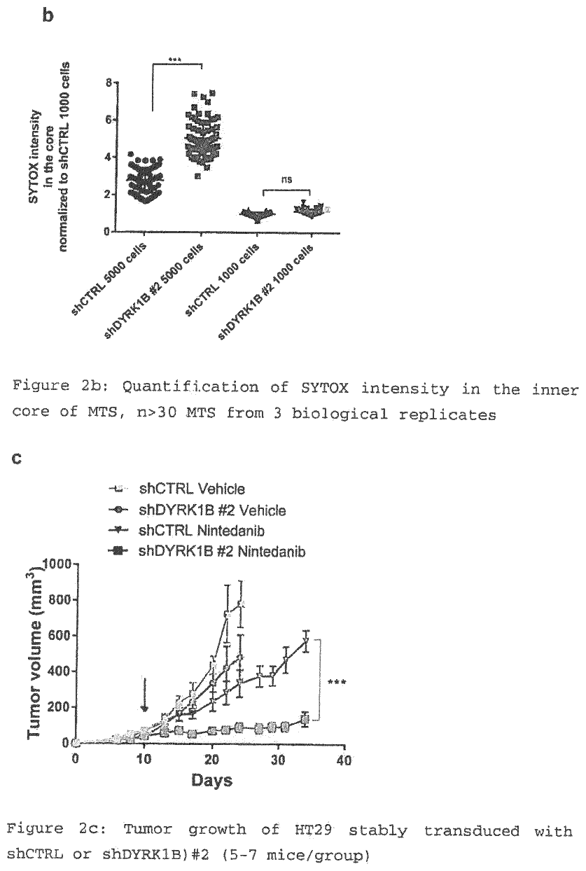

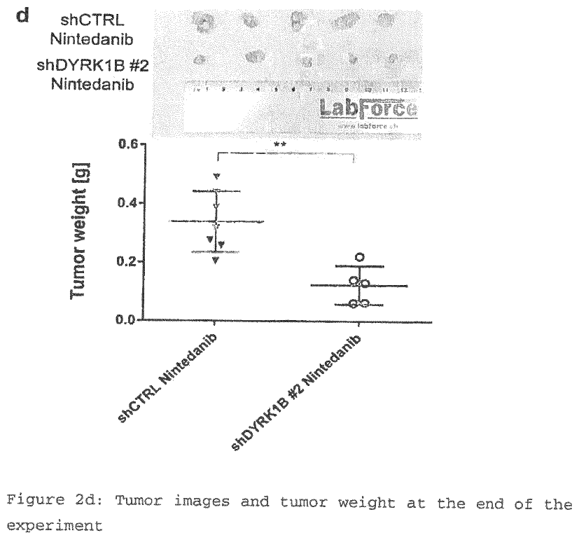

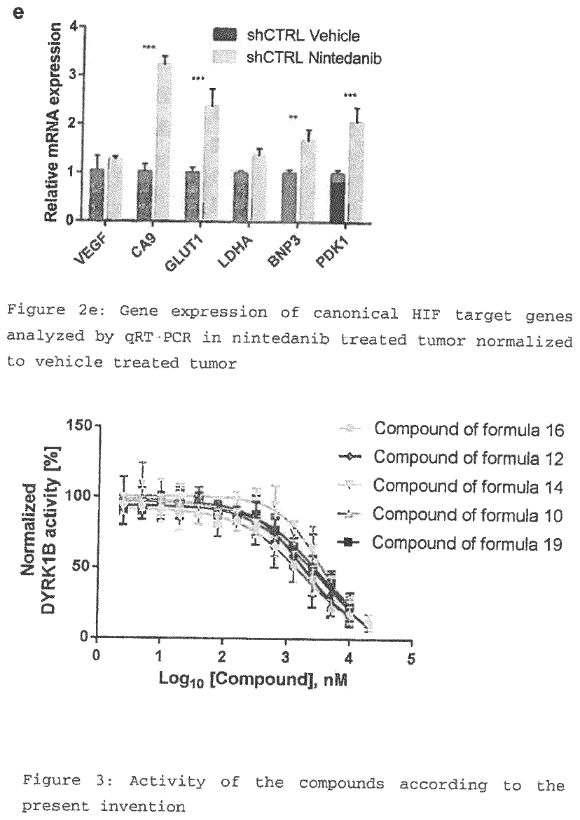

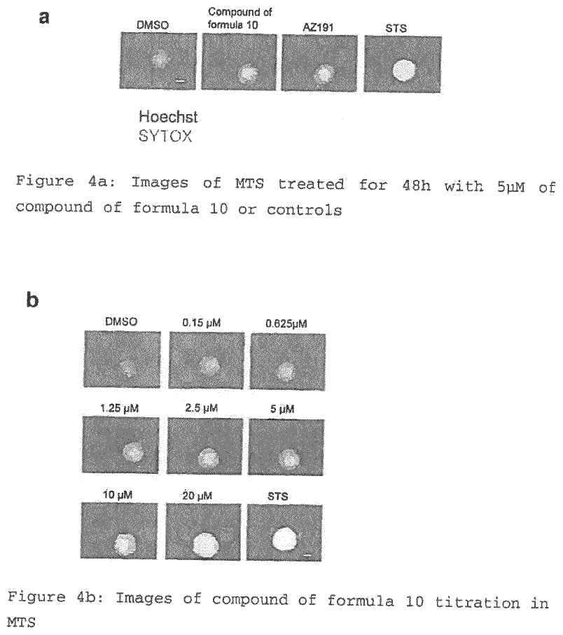

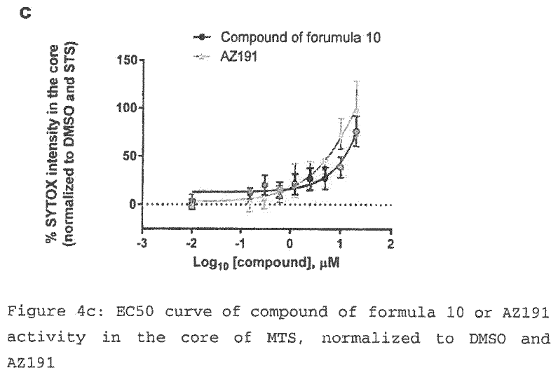

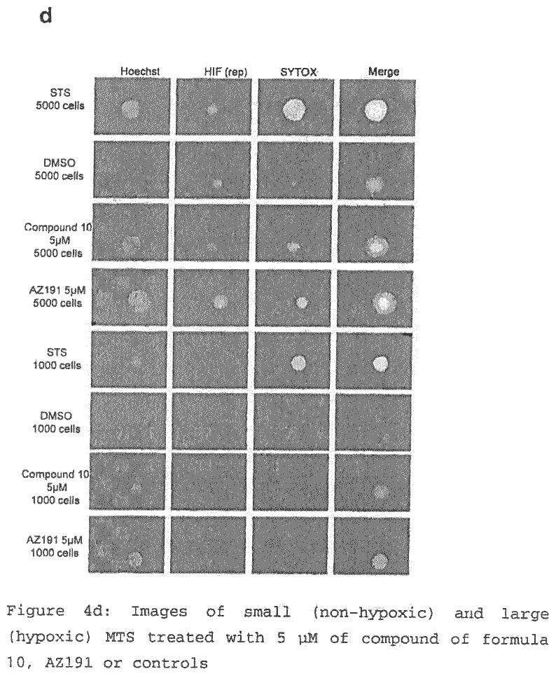

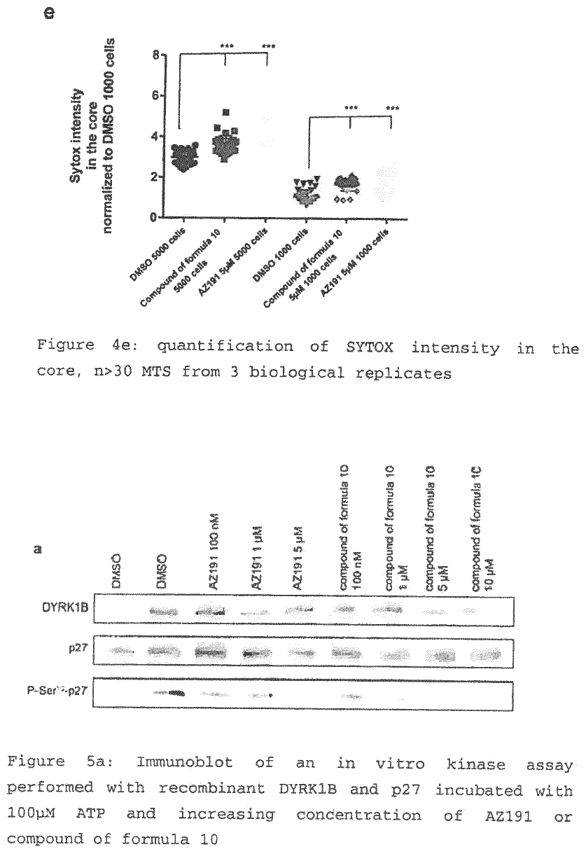

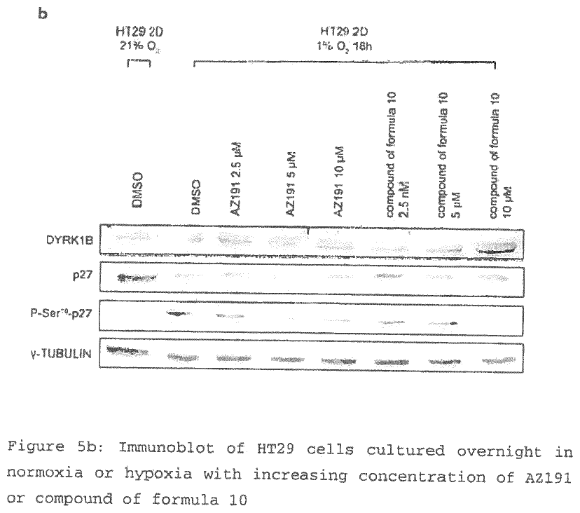

Embodiments of the present invention are illustrated by the following Examples, but not limited thereto.

### EXAMPLES

### Cell Culture, shRNA

All cell lines were obtained from ATCC. WM266-4 were cultured in DMEM, HT29 and DLD1 in RPMI. Medium were supplemented with 10% fetal calf serum (FSC) and 1% penicillin-streptomycin. AZ191 was acquired from Selleck Chemicals and STS from sigma. shRNA viral particles were produced following the broad institute protocol. shRNA used in this study were shCTRL (pLKO.1-puro Luciferase: SHC007), shDYRK1B#1 (TRCN0000002142) or shDYRK1B#2 (TRCN0000002139).

### Generation of HIF Reporter Cell Lines

WM266-4 or HT29 were transduced with lentiviral particles of the HIF reporter HBR-6U (Addgene 42621). To select for cells displaying good activation of the reporter under hypoxia, cells were culture overnight in a hypoxia chamber (SCI-tive Baker Ruskinn) at 1% of oxygen. The next day, cells showing a strong YFP signal were FACS sorted (Aria BL1) and put back in culture.

### MTS Culture

WM266-4-HRE, HT29-HRE and DLD1 were transduced with shCTRL, shDYRK1B#1 or shDYRK1B#2 and selected with puromycin. For MTS formation, 250 μl of the pools cell suspension was dispensed in ULA plates (WM266-4-HRE: 80000 cells/ml, HT29-HRE: 20000 cells/ml DLD1: 40000 cells/ml) and spun at 700 g for 5 min. For rescue experiment, 50 μl of rescue construct viral particles were added to the cell suspension in the ULA plate. For hypoxia experiments, the plate was placed 24 h in a hypoxia chamber at 1% O2.

### MTS Staining and Analysis

After 72h hours in culture, 10 μl of a mix of SYTOX Orange (S11368) (1:1000) and Hoechst 33258 (1 μM) diluted in DMEM was added to each well of the ULA plate and incubated overnight before image acquisition. MTS were imaged with a 5× objective on Zeiss observer Z.1 microscope equipped with an automated stage, with Hoechst channel used for autofocusing. For each well, images of Hoechst, YFP and SYTOX channels were acquired in the middle of the MTS. Images were analyzed with a custom ImageJ script. Briefly, Hoechst channel images were used to generate a mask of the MTS, which was then scaled to 1:3 and transferred to the SYTOX channel to measure the pixel intensity in the core area of the MTS.

### Compound Treatment of MTS

HT29-HRE solution (20,000 cells/ml) was dispensed in ULA plates (250 μl/well) and spun at 700 g for 5 min. After 48 h, MTS were treated with 10 μl of serial dilutions of AZ191, compound of formula 10 DMSO or STS. The next day a mix of Hoechst and SYTOX was added to the wells. MTS were then processed as described above.

### Quantitative Real-time PCR

RNA was extracted using NucleoSpin® RNA Machinery Nagel according to the manufacturer's instructions. RNA yield and purity were measured using a nanodrop. 1 μg of RNA was used to produce cDNA using the High-Capacity RNA-to-cDNA™ Kit (Thermo catalog number 4387406) following manufacturer's instructions. qPCR reactions were carried out with KAPA SYBR FAST (Axon Lab AG) according to manufacturer's instructions and ran on the LightCycler® 480 Real-Time PCR System. Results were analyzed with LightCycler® 480 Software and normalized to TPB expression.

### Cloning of Rescue Construct

pENTR221 (DQ895747) was mutated with Phusion Site-Directed Mutagenesis Kit (F541) according to manufacturer's instructions. The first two codons of shDYRK1B#2 binding region were mutated with the following primers: Forward (GGACCTCATCAAAACTTACAAGCACATC) reverse (ACAGAGAGCTTACGCAGCGGGGC). Mutated DYRK1B was then cloned into pLX301-DEST by LR recombination using the Gateway® system.

### Western Blot

Cells were lysed in lysis buffer (50 mM Tris-HCl pH 7.5, 250 mM NaCl, 0.5% NP-40, 5 mM NaCl, 5 mM NaF, 5 mM EDTA, 0.5 mM EGTA). Protein were separated in 10% SDS/PAGE gels, as previously described. Ser¹⁰-p27 (sc-12939-R) (1:500) and DYRK1B (sc-390417) (1:200) antibodies were purchased from Santa-Cruz, γ-TUBULIN (ab11316) (1:5000) from Abcam, p27 (610242) (1:500) from BD Biosciences and V5 (R960-25) from Life Technology.

### p27-GST Bacculovirus Production

Insect cells Sf9 were grown in a flask under constant agitation at 28° C. in Sf-900 II SFM medium. For protein expression, 100 ml of Sf9 cells (˜2*10⁶/ml) were transduced with 1 ml of GST-p27 bacculovirus and grown for 3 days. Finally, cells were collected by centrifugation, washed with PBS and lysed in 40 ml TNN. The lysate was filtered through a 0.45 μm filter and loaded on GSTrap™ 4B 1 ml column (GE Healthcare) using a peristaltic pump. Purification steps were carried out according to manufacturer's instructions. Several fractions of elution were collected and analyzed by WB against a bovine serum albumin (BSA) standard to determine the protein concentration.

### In Vitro Kinase Assay

28 ng of GST-DYRK1B (ThermoFisher, NP_004705) and 100 ng of GST-p27 were mixed in 45 μl of kinase buffer (50 mM HEPES (pH 7.5), 1 mM DTT, MgCl2, 0.004% Triton X-100). Serial dilution of AZ191, compound of formula 10 or DMSO were added to the mix and incubated for 5 min at room temperature. 100 μM of ATP was added to start the kinase reaction which was carried out with constant shaking at 30° C. for 30 min. The reaction was terminated by addition of Laemmli buffer and boiled 5 min at 95° C. Finally, the proteins were separated by SDS page and detected by immunoblotting.

### IC50 Determination of DYRK1B Inhibitors

GST-DYRK1B (5 nM) and DYRKtide (50 μM) (Lucerna-Chem AG, D96-58) diluted in kinase buffer were dispensed in white 384 well plate (3572, Corning, N.Y., USA) (2 μl/well). Following compound dispensing (1 μl), 2 μl of ATP (final concentration 100 μM) was added and incubated for 30 min at room temperature. After that, 5 μl of ADP-Glo™ reagent was dispensed and incubated for 40 min at room temperature. Finally, 10 ul of Kinase Detection Reagent was dispensed and incubated for 30 min before measuring the plate on a Tecan infinite M1000pro plate reader (Luminescence mode).

### Animal Studies

2 month old athymic mice (BALB/cAnNRj-Foxn1nu/nu) were subcutaneously injected bilaterally with stable pools of HT29-HRE shCTRL (left flank), shDYRK1B#2 (right flank), (2*10⁶ cells/flank) using a 28G needle. Tumor size was measured with a caliper and the volume was calculated according to the formula I²*L/2, where I is the shortest measured diameter of the tumor and L the largest. When tumors reached an average of 50 mm³, mice were separated into 2 groups (Vehicle treatment and nintedanib treatment). Vehicle and nintedanib (50 mg/kg nintedanib diluted in 0.5% Natrosol (w/v)) were administered daily by oral gavage (o.g).

### Statistical Analysis

Data are displayed as mean±standard deviation (SD) or mean±standard error mean (SEM), as indicated in the figure legend. Statistical analysis was performed with unpaired two-tailed Student's t-test or one-way ANOVA for multiple comparisons. A p-value lower than 0.05 was considered significant (* p<0.05, ** p<0.01, *** p<0.001, **** p<0.0001).

### IC₅₀ Values for Selected Examples

| Compound according to the invention | IC₅₀ [nM] |
|---|---|
| 4-[1-(2-fluorophenyl)-1H-pyrazol-4-yl]-N-[(3-methyl-2- | 3520 |
| pyridinyl)methyl]-2-pyrimidinamine | |
| 5-({4-[1-(2-fluorophenyl)-1H-pyrazol-4-yl]pyrimidin-2-yl} | 2005 |
| amino)pentan-1-ol | |
| 4-[1-(2-methoxyphenyl)-1H-pyrazol-4-yl]-N-[(5-methyl-2- | 10325 |
| pyrazinyl)methyl]-2-pyrimidinamine | |
| N-(2,2-difluoroethyl)-4-[1-(2-fluorophenyl)-1H-pyrazol-4-yl] | 6982 |
| pyrimidin-2-amine | |
| N-{4-[1-(2-fluorophenyl)-1H-pyrazol-4-yl]pyrimidin-2-yl} | 3968 |
| pentane-1,5-diamine | |
| Reference Compound | IC₅₀ [nM] |
| N-{4-[1-(4-fluorophenyl)-1H-pyrazol-4-yl]pyrimidin-2-yl} | 15614 |
| pentane-1,5-diamine | |

1. Compound of formula (I) 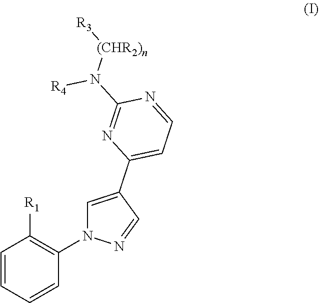
or a pharmaceutically acceptable salt thereof, wherein
R₁ is selected from fluoro, methoxy and ethoxy,
each R₂ is independently selected from hydrogen, fluorine and methyl,
n is 0, 1, 2, 3, 4 or 5,
R₃ is selected from hydrogen, fluorine, amino, hydroxy, and a five or six membered substituted or unsubstituted ring system which may be aromatic or aliphatic, comprising 1 or 2 heteroatoms selected from the group consisting of nitrogen and oxygen,
R₄ is hydrogen or methyl,
for use as a therapeutically active substance.

2. The compound of claim 1, wherein the compound is of formula (Ia) 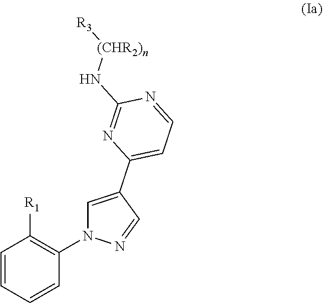
or a pharmaceutically acceptable salt thereof, wherein
R₁ is fluoro, methoxy or ethoxy,
R₂ is hydrogen or methyl,
n is 0, 1 or 2,
R₃ is hydrogen, or a five or six membered substituted or unsubstituted ring system which may be aromatic or aliphatic, comprising 1 or 2 heteroatoms selected from the group consisting of nitrogen and oxygen,
for use as a therapeutically active substance.

3. Compound according to claim 1 or 2 for use in the treatment of cancer.

4. Compound according to any of the preceding claims, wherein the compound is selected from the group consisting of
4-(1-(2-fluorophenyl)-1H-pyrazole-4-yl)-N-(pyridine-4-ylmethyl)pyrimidine-2-amine,
4-(1-(2-methoxyphenyl)-1H-pyrazole-4-yl)-N-(pyridine-4-ylmethyl)pyrimidine-2-amine,
4-(1-(2-fluorophenyl)-1H-pyrazole-4-yl)-N-(tetrahydro-2H-pyran-4-yl)pyrimidine-2-amine,
4-(1-(2-methoxphenyl)-1H-pyrazole-4-yl)-N-(tetrahydro-2H-pyran-4-yl)pyrimidine-2-amine,
N-ethyl-4-(1-(2-fluorophenyl)-1H-pyrazole-4-yl)pyrimidine-2-amine,
N-ethyl-4-(1-(2-methoxyphenyl)-1H-pyrazole-4-yl)pyrimidine-2-amine,
4-(1-(2-fluorophenyl)-1H-pyrazole-4-yl)-N-(1-(pyridine-4-yl) propane-2-yl)pyrimidine-2-amine,
4-(1-(2-methoxyphenyl)-1H-pyrazole-4-yl)-N-(1-(pyridine-4-yl) propane-2-yl)pyrimidine-2-amine,
N-((3,5-dimethyl-1H-pyrazole-4-yl)methyl)-4-(1-(2-fluorophenyl)-1H-pyrazole-4-yl)pyrimidine-2-amine, and
N-((3,5-dimethyl-1H-pyrazole-4-yl)methyl)-4-(1-(2-methoxyphenyl)-1H-pyrazole-4-yl)pyrimidine-2-amine.

5. Compound according to any of the preceding claims, wherein the compound is selected from the group consisting of
4-(1-(2-fluorophenyl)-1H-pyrazole-4-yl)-N-(pyridine-4-ylmethyl)pyrimidine-2-amine,
4-(1-(2-fluorophenyl)-1H-pyrazole-4-yl)-N-(tetrahydro-2H-pyran-4-yl)pyrimidine-2-amine,
N-ethyl-4-(1-(2-fluorophenyl)-1H-pyrazole-4-yl)pyrimidine-2-amine,
4-(1-(2-fluorophenyl)-1H-pyrazole-4-yl)-N-(1-(pyridine-4-yl) propane-2-yl)pyrimidine-2-amine, and
N-((3,5-dimethyl-1H-pyrazole-4-yl)methyl)-4-(1-(2-fluorophenyl)-1H-pyrazole-4-yl)pyrimidine-2-amine, preferably 4-(1-(2-fluorophenyl)-1H-pyrazole-4-yl)-N-(pyridine-4-ylmethyl)pyrimidine-2-amine.

6. Compound according to any one of claims 1 to 3, wherein the compound is selected from the group consisting of
4[1-(2-fluorophenyl)-1H-pyrazol-4-yl]-N-[(3-methyl-2-pyridinyl)methyl]-2-pyrimidinamine,
5-({4-[1-(2-fluorophenyl)-1H-pyrazol-4-yl]pyrimidin-2-yl}amino)pentan-1-o,
N-(2,2-difluoroethyl)-4-[1-(2-fluorophenyl)-1H-pyrazol-4-yl]pyrimidin-2-amine, and
N-{4-[1-(2-fluorophenyl)-1H-pyrazol-4-yl]pyrimidin-2-yl}pentane-1,5-diamine.

7. Compound according to any one of claims 1 to 3, wherein the compound is selected from the group consisting of
5-({4-[1-(2-fluorophenyl)-1H-pyrazol-4-yl]pyrimidin-2-yl}amino)pentan-1-ol and
N-{4-[1-(2-fluorophenyl)-1H-pyrazol-4-yl]pyrimidin-2-yl}pentane-1,5-diamine.

8. Compound according to any of the preceding claims, wherein said tumor is a solid or a non-solid tumor.

9. Compound according to any of the preceding claims, wherein said tumor is treatment resistant, preferably drug-resistant.

10. Compound according to any of the preceding claims, wherein said tumor comprises hypoxic tumor cells and/or glycolytic cancer cells.

11. Compound according to any of the preceding claims, wherein said tumor comprises dormant/arrested cancer cells.

12. Compound according to any of the preceding claims, wherein said tumor comprises tumor stem cells.

13. Compound according to any of the preceding claims, wherein growth of said tumor is associated with overexpression of dual specificity tyrosine-phosphorylation-regulated kinase 1B (DYRK1B) (SEQ. ID. NO. 1).

14. A pharmaceutical composition comprising a compound as defined in any one of claims 1 to 13 and a pharmaceutically acceptable excipient.

15. Pharmaceutical combination comprising a compound as defined in claims 1, 4 and 5 and an anti-angiogenic inhibitor and/or a radio- and/or chemotherapeutic drug and/or cell cycle inhibitor.

16. Pharmaceutical combination according to claim 15, wherein the antiangiogenic inhibitor is selected from the group consisting of bevacizumab, ziv-aflibercept, sorafenib, sunitinib, axitinib, nintedanib, regorafenib, pazobanib, cabozantinib, vandetanib, and thalidomide.

17. Pharmaceutical combination according to claim 15, wherein the radio- and/or chemotherapeutic drug is selected from the group consisting of group consisting of alkylating agents, vinca alkaloids, aromatase inhibitors, selective estrogen receptor modulators, topoisomerase I inhibitors, topoisomerase II inhibitors, microtubule stabilizing and disrupting agents, tubulin binding agents, proteosome inhibitors, mTOR inhibitors and conjugated antibodies.

18. Pharmaceutical combination according to claim 15, wherein the cell cycle inhibitor is an inhibitor of a member of the cip/kip family or INK4a/ARF family, in particular an inhibitor of p21, p27, p57, p16, CDK4, p14, p53, CDK6, Cdc25.

19. Pharmaceutical combination according to claim 17, wherein the radio- and chemotherapeutic drug is preferably selected from the group consisting of bleomycin, dactinomycin, doxorubicin, epirubicin, idarubicin, mitomycin, mitoxantron, irinotecan, topotecan, amsacrin, daunorubicin, etoposid, anagrelid, azacitidin, capecitabin, clofarabin, cytarabin, fludarabin, 5-fluorouracil, gemcitabin, mercaptopurin, nelarabin, tioguanin, cisplatin, carboplatin, oxaliplatin, bendamustin, busulfan, chlorambucil, chlormethin, cyclophosphamid, dacarbazin, ifosfamid, lomustin, melphalan, pro-carbazin, streptozocin, temozolomid, carmustin (-), methotrexat, pemetrexed, raltitrexed, cabazitaxel, docetaxel, paclitaxel, vinblastin, vincristin, vindesin, vinorelbin, eribulin, cladribin, podophyllotoxin, hydroxycarbarnid, and ixabepilon.

20. Use of a compound as defined in claims 1, 4 and 5 as inhibitor of dual specificity tyrosine-phosphorylation-regulated kinase 1B (DYRK1B) (SEQ. ID. NO. 1) and/or DYRK1A (SEQ ID NO 4).


SEQUENCE LISTING (4 sequences)

SEQ ID NO: 1 - protein, 629 aa, Homo sapiens
  DYR1B_HUMAN Dual specificity tyrosine- phosphorylation-regulated kinase 1B
MAVPPGHGPFSGFPGPQEHTQVLPDVRLLPRRLPLAFRDATSAPLRKLSVDLIKTYKHINEVYYAKKKRRAQQAPPQDSSNKKEKKVLNHGYDDDNHDYIVRSGERWLERYEIDSLIGKGSFGQVVKAYDHQTQELVAIKIIKNKKAFLNQAQIELRLLELMNQHDTEMKYYIVHLKRHFMFRNHLCLVFELLSYNLYDLLRNTHFRGVSLNLTRKLAQQLCTALLFLATPELSIIHCDLKPENILLCNPKRSAIKIVDFGSSCQLGQRIYQYIQSRFYRSPEVLLGTPYDLAIDMWSLGCILVEMHTGEPLFSGSNEVDQMNRIVEVLGIPPAAMLDQAPKARKYFERLPGGGWTLRRTKELRKDYQGPGTRRLQEVLGVQTGGPGGRRAGEPGHSPADYLRFQDLVLRMLEYEPAARISPLGALQHGFFRRTADEATNTGPAGSSASTSPAPLDTCPSSSTASSISSSGGSSGSSSDNRTYRYSNRYCGGPGPPITDCEMNSPQVPPSQPLRPWAGGDVPHKTHQAPASASSLPGTGAQLPPQPRYLGRPPSPTSPPPPELMDVSLVGGPADCSPPHPAPAPQHPAASALRTRMTGGRPPLPPPDDPATLGPHLGLRGVPQSTAASS

SEQ ID NO: 2 - DNA, 28 bp, Artificial Sequence
  primer (forward)
ggacctcatcaaaacttacaagcacatc

SEQ ID NO: 3 - DNA, 23 bp, Artificial Sequence
  primer (reverse)
acagagagcttacgcagcggggc

SEQ ID NO: 4 - protein, 763 aa, Homo sapiens
  DYRK1A_human dual specificity tyrosine- phosphorylation-regulated kinase 1A
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFTAAVQAMDCETHSPQVRQQFPAPLGWSGTEAPTQVTVETHPVQETTFHVAPQQNALHHHHGNSSHHHHHHHHHHHHHGQQALGNRTRPRVYNSPTNSSSTQDSMEVGHSHHSMTSLSSSTTSSSTSSSSTGNQGNQAYQNRPVAANTLDFGQNGAMDVNLTVYSNPRQETGIAGHPTYQFSANTGPAHYMTEGHLTMRQGADREESPMTGVCVQQSPVASS